(19) United States
(12) Patent Application Publication
(10) Pub. No.: US 2010/0267949 A1
(43) Pub. Date: Oct. 21, 2010

(54) Method of Synthesizing 6,7-Substituted 4-Anilino Quinazoline

(76) Inventors: Kae-Shyang Shih, Taipei (TW); Yu-Jung Hsieh, Taipei (TW); Ching-Wei Liu, Taipei (TW)

Correspondence Address: LanWay IPR Services, P.O. Box 220746, Chantilly, VA, 20153, US

(21) Appl. No.: 12/464,868

(22) Filed: May 13, 2009

(30) Foreign Application Priority Data
Apr. 16, 2009 (CN) 200910133962.3

(51) Int. Cl. C07D 413/02; C07D 239/72

(52) U.S. Cl. 544/119; 544/283

(57) ABSTRACT

A method of synthesizing 6,7-substituted 4-anilino quinazoline employs 3,4-substituted benzoic acid as an initial reactant, and the 6,7-substituted 4-anilino quinazoline is obtained by an esterifying step, a nitrating step, a reducing step, a cyclizing step, and an one-pot reaction. In the above method, the initial reactant has low cost and yield. of the 6,7-substituted 4-anilino quinazoline is high, therefore, production cost can be reduced effectively, and competitive power of the product of the 6,7-substituted 4-anilino quinazoline can be improved.

### REFERENCE TO RELATED APPLICATION

The present application claims the right of priority based on China Application Serial Number 200910133962.3, filed on Apr. 4, 2009, the disclosure of which is incorporated herein by reference in its entirety.

### BACKGROUND

1. Technical Field

The present invention relates to a method of synthesizing quinazoline derivative, and more particularly to a method of synthesizing 6,7-substituted 4-anilino quinazoline.

2. Description of the Related Art

Epidermal growth factor receptor (EGFR) plays an important role in the development of cancer. 6,7-substituted 4-anilino quinazoline that has been widely used clinically is mainly utilized to cure non-small-cell lung cancer (NSCLC), such as Gefitinib, Erlotinib, and Vandetanib for curing lung cancer and Thyroid cancer in the market. In addition, the 6,7-substituted 4-anilino quinazoline is utilized in the Phase II clinical trials of Myeloid leukemia and Myelodysplasia, such as Tandutinib. The 6,7-substituted 4-anilino quinazoline is represented by formula as follows:

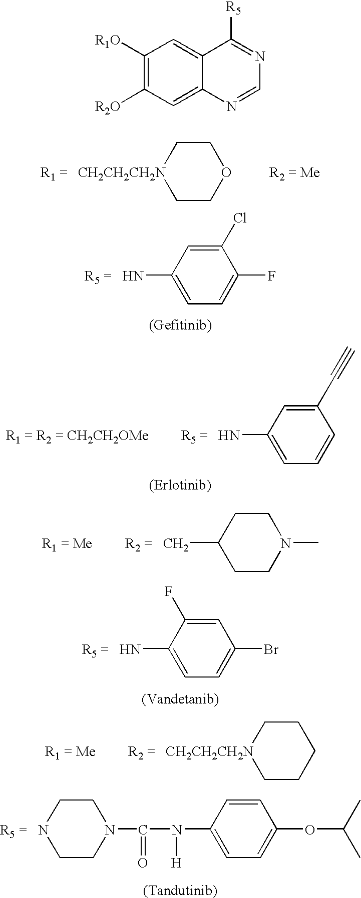

Some methods of synthesizing 6,7-substituted 4-anilino quinazoline have been disclosed in literature as follows.

(1) WO 2004024703

Isovanilln is employed as an initial reactant, aldehyde group of Isovanilln is converted into nitrile group (CN) by using HCO₂Na/HCO₂H/(HCONH₂)₂H₂SO₄; the nitrile group is converted into amide group by an O-alkylation step, a nitrating step and a reducing step; then a cyclizing step with HCO₂H/HCONH₂ is carried out to yield quinazoline; and finally, Gefitinib is obtained by a chlorinating step and an aniline derivative substituting step.

(2) WO 9633980

6,7-dimethoxy-3,4-dihydroquinazolin-4-one is employed as an initial reactant; a demethylating step by using L-methionine and a protecting step by using Ac₂O are carried out at a sixth position; then a chlorinating step and an aniline derivative substituting step are carried out for removing the Ac₂O; and finally, Gefitinib is obtained by an O-alkylation step. P (3) Organic Process Research & Development 2007, 11, 813-816

Isovanilln is employed as an initial reactant; aldehyde group of Isovanilln is converted into nitrile group; after an O-alkylation step, a nitrating step and a reducing step, N,N-dimethylformamidine derivative is yielded with the help of dimethylformamide-dimethylacetal (DMF-DMA); and finally, Gefitinib is obtained by a Dimroth rearrangement reaction with aniline derivative compounds. In addition, Erlotinib can be obtained by using the same method as above.

(4) CN 1733738

3,4-dimethoxybenzoic acid is employed as an initial reactant, and after a nitrating step, a demethylating step, a reducing step, a cyclizing step, a chlorinating step and an aniline derivative substituting step are carried out, Gefitinib is obtained by an O-alkylation step.

(5) CN 101148439A

Methyl 3-hydroxy-4-methoxybenzoate is employed as an initial reactant, and Gefitinib is obtained by an O-alkylation step, a nitrating step, a reducing step, a cyclizing step, a chlorinating step and an aniline derivative substituting step.

However, there are some shortcomings in the above method of synthesizing the 6,7-substituted 4-anilino quinazoline. For example, high cost of raw material, complicated reaction steps, unstable intermediate product and low yield.

Therefore, a new method of synthesizing 6,7-substituted 4-anilino quinazoline is desired in order to overcome the above-described shortcomings.

### BRIEF SUMMARY

The present invention relates to method of synthesizing 6,7-substituted 4-anilino quinazoline.

A method of synthesizing 6,7-substituted 4-anilino quinazoline is provided. The 6,7-substituted 4-anilino quinazoline is represented by formula as follows,

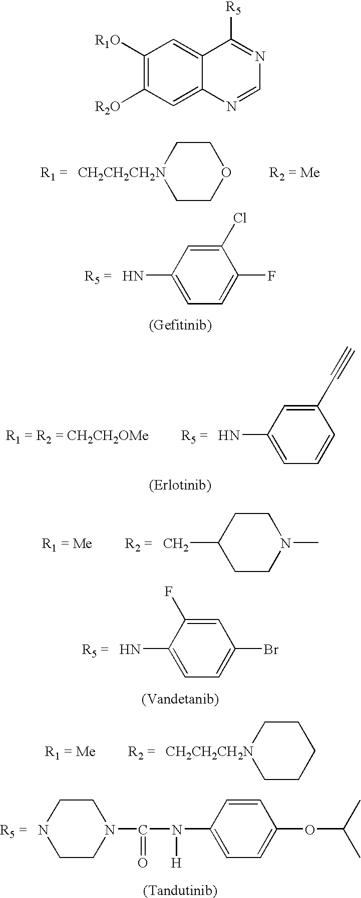

The method includes: (a) employing 3,4-substituted benzoic acid as an initial reactant, and a first esterifying step is performed on the 3,4-substituted benzoic acid to yield alkyl 3,4-substituted benzoate, wherein each substituted group of the 3,4-substituted benzoic acid or the alkyl 3,4-substituted benzoate is one of an alkyl group and a hydroxyl group, and oxygen in the substituted groups is defined as a first oxygen and a second oxygen correspondingly; (b) nitrating the alkyl 3,4-substituted benzoate to yield alkyl 2-nitro-4,5-substituted benzoate; (c) reducing the alkyl 2-nitro-4,5-substituted benzoate to yield alkyl 2-amino-4,5-substituted benzoate; (d) cyclizing the alkyl 2-amino-4,5-substituted benzoate to yield 6,7-substituted quinazolin-4-one; and (e) performing a one-pot reaction on the 6,7-substituted quinazolin-4-one to yield 6,7-substituted 4-anilino quinazoline, wherein the one-pot reaction comprises a chlorinating step and an aniline derivative substituting step.

In the above method, the initial reactant has low cost and yield of the 6,7-substituted 4-anilino quinazoline is high, therefore, production cost can be reduced effectively, and competitive power of the product of the 6,7-substituted 4-anilino quinazoline can be improved.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 is a flow chart of a method of synthesizing 6,7-substituted 4-anilino quinazoline according to an exemplary embodiment of the present invention, showing formulas in different steps.

FIG. 2 is a flow chart of a method of synthesizing 6,7-substituted 4-anilino quinazoline according to another exemplary embodiment of the present invention, showing formulas in different steps.

FIG. 3 is a flow chart of a method of synthesizing 6,7-substituted 4-anilino quinazoline according to further another exemplary embodiment of the present invention, showing formulas in different steps.

### DETAILED DESCRIPTION

A method of synthesizing 6,7-substituted 4-anilino quinazoline of the present invention employs 3,4-substituted benzoic acid as an initial reactant. In an embodiment, the method of the synthesizing 6,7-substituted 4-anilino quinazoline without a hydrolysis-demethylation step, and the 6,7-substituted 4-anilino quinazoline can be obtained by an esterifying step, an O-alkylation step, a nitrating step, a reducing step, a cyclizing step and an one-pot reaction. In an alternative embodiment, the method of synthesizing the 6,7-substituted 4-anilino quinazoline includes a hydrolysis-demethylation step, and the 6,7-substituted 4-anilino quinazoline can be obtained by an esterifying step, an O-alkylation step or a nitrating step, a hydrolysis-demethylation step, an esterifying step, an O-alkylation step, a reducing step, a cyclizing step and an one-pot reaction. The 6,7-substituted 4-anilino quinazoline synthesized by the above methods can be one of Gefitinb, Erotinib, Vandetanib and Tandutinib, and the 6,7-substituted 4-anilino quinazoline is represented by formula as follows:

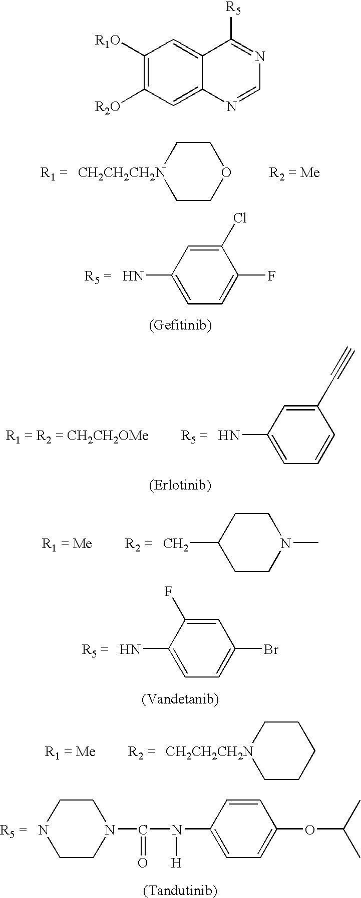

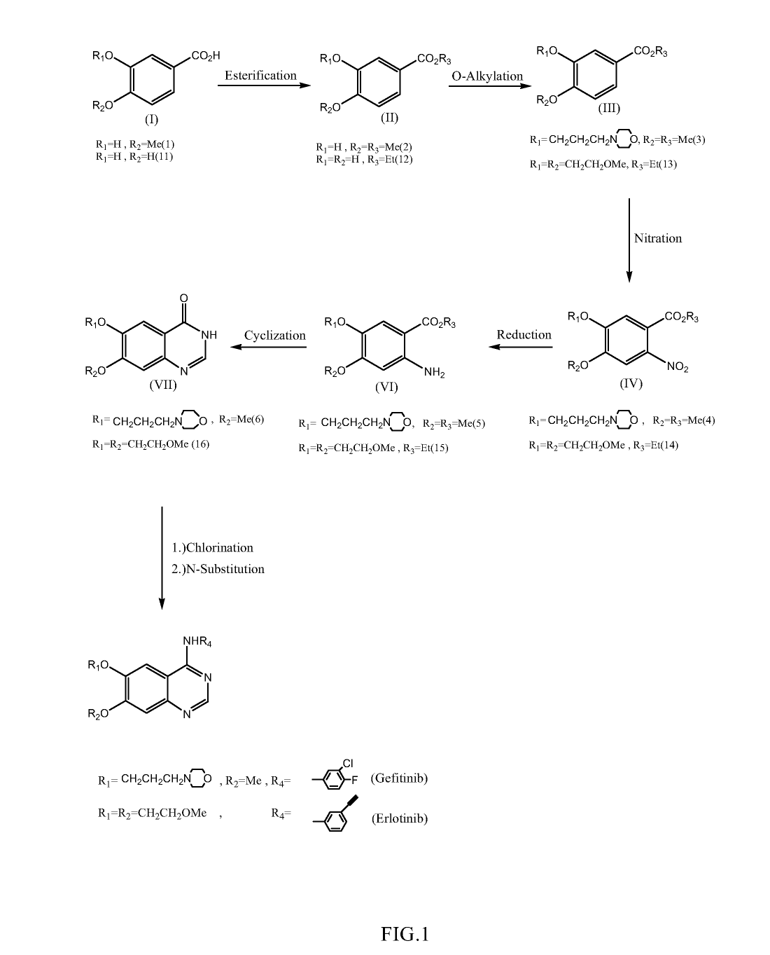

FIG. 1 is a flow chart of a method of synthesizing 6,7-substituted 4-anilino quinazoline according to an exemplary embodiment of the present invention, showing formulas in different steps. Referring to FIG. 1, in this embodiment, 3-hydroxy-4-methoxybenzoic acid 1 or 3,4-dihydroxybenzoic acid 11 is employed as initial reactant, and the 6,7-substituted 4-anilino quinazoline can be obtained by an esterifying step, an O-alkylation step, a nitrating step, a reducing step, a cyclizing step and an one-pot reaction.

A process of the method of this embodiment would be described as follows.

(1) Esterifying step:

By the Fischer esterifying step or with SOCl₂ in alcohol solvent, the 3,4-substituted benzoic acid I, such as 3-hydroxy-4-methoxybenzoic acid 1 or 3,4-dihydroxybenzoic acid 11 can be converted into compound II. The compound II is methyl 3-hydroxy-4-methoxybenzoate 2 or ethyl 3,4-dihydroxy benzoate 12 correspondingly.

(2) O-alkylation Step:

Methyl 3-hydroxy-4-methoxybenzoate 2 or ethyl 3,4-dihydroxy benzoate 12 can be respectively dissolved in organic solvent including dimethylformamide, CH₃CN and acetone or in organic solvent and water, with 3-morpholinopropoxy chloride and 2-bromoethyl methyl ether, and converted into compound III under the condition of weak base or KI, at 50150° C. The compound III is methyl 4-methoxy-3-(3-morpholinopropoxy)benzoate 3 or ethyl 3,4-dimethoxyethoxybenzoate 13 correspondingly.

(3) Nitrating Step:

Methyl 4-methoxy-3-(3-morpholinopropoxy)benzoate 3 or ethyl 3,4-dimethoxyethoxybenzoate 13 can be respectively dissolved in acetic acid, then 70% H₂SO₄ and 45% HNO₃ can be added, and then compound IV can be obtained at 25˜150° C. by the nitrating step. The compound IV is methyl 2-nitro-4-methoxy-5-(3-morpholinopropoxy)benzoate 4 or ethyl 2-nitro-4,5-dimethoxyethoxybenzoate 14 correspondingly.

(4) Reducing Step:

Methyl 2-nitro-4-methoxy-5-(3-morpholinopropoxy)benzoate 4 or ethyl 2-nitro-4,5-dimethoxyethoxybenzoate 14 can be respectively dissolved in alkali solution with Na₂S₂O₄ to be reduced to yield compound VI; or the reducing step can be performed to yield the compound VI, by employing 10% Pd—C as a catalytic agent, using hydrogen gas under a pressure of 30˜60 psi and organic solvent selected from one of ethyl acetate and alcohol at 25˜100° C. The compound VI is methyl 2-amino-5-methoxy-4-(3-morpholinopropoxy)benzoate 5 or ethyl 2-amino-4,5-dimethoxyethoxybenzoate 15 correspondingly.

(5) Cyclizing Step:

methyl 2-amino-5-methoxy-4-(3-morpholinopropoxy)benzoate 5 or ethyl 2-amino-4,5-dimethoxyethoxybenzoate can be respectively cyclized to yield compound VII by adding HCO₂NH₄ and HCONH₂ therein at 80˜200° C. The compound VII is 7-methoxy-6-(3-morpholinopropoxy)-3,4-dihydroquinazolin-4-one 6 or 6,7-bis(2-methoxyethoxy)quinazolin-4-one 16 correspondingly.

(6) One-Pot Reaction (a Chlorinating Step and an Aniline Derivative Substituting Step):

7-methoxy-6-(3-morpholinopropoxy)-3,4-dihydroquinazolin-4-one 6 or 6,7-bis(2-methoxyethoxy)quinazolin-4-one 16 can be respectively added in organic solvent, then be chlorinated by using 2˜20 equivalents of PCl₅, POCl₃ and SOCl₂, and then chlorinate that was obtained directly and aniline derivative can be added in alcohol to carry out the aniline derivative substituting step. As such, the product of Gefitinib or Erlotinib whose purity is greater than 99.7% can be obtained correspondingly.

In the method of synthesizing the 6,7-substituted 4-anilino quinazoline of the exemplary embodiment, 3-hydroxy-4-methoxybenzoic acid 1 or 3,4-dihydroxybenzoic acid 11 is employed as initial reactant, and the 6,7-substituted 4-anilino quinazoline is synthesized without the hydrolysis-demethylation step.

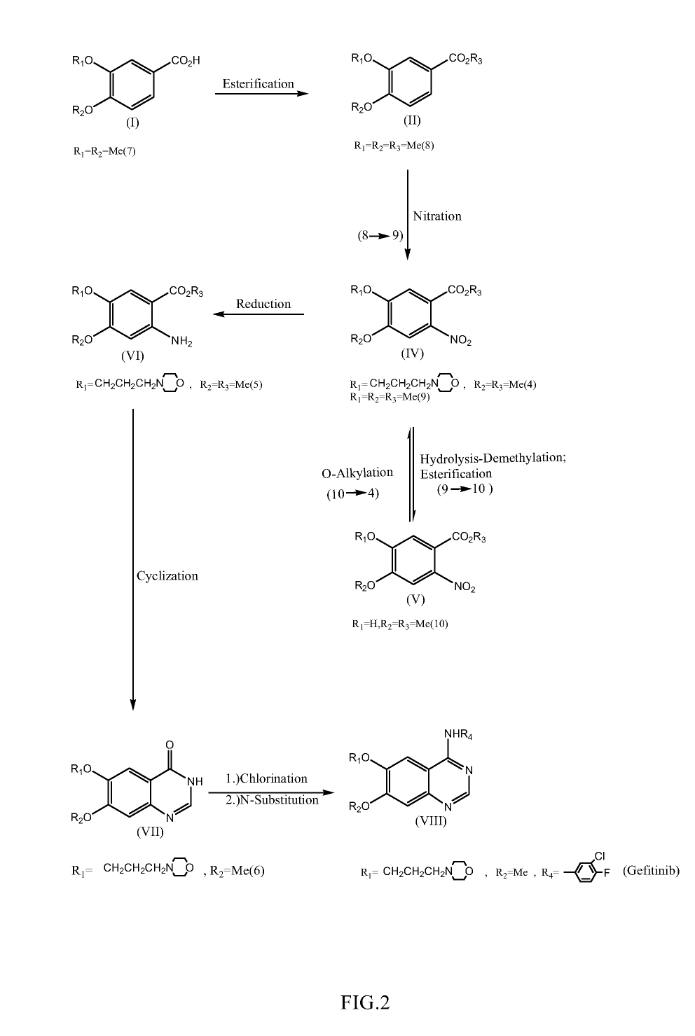

FIG. 2 is a flow chart of a method of synthesizing 6,7-substituted 4-anilino quinazoline according to another exemplary embodiment of the present invention, showing formulas in different steps. Referring to FIG. 2, in this embodiment, 3,4-dimethoxybenzoic acid 7 is employed as initial reactant, and the 6,7-substituted 4-anilino quinazoline can be obtained by an esterifying step, a nitrating step, a hydrolysis-demethylation step, an esterifying step, an O-alkylation step, a reducing step, a cyclizing step and an one-pot reaction.

(1) Esterifying Step:

By the Fischer esterifying step or with SOCl₂ in alcohol solvent, 3,4-dimethoxybenzoic acid 7 can be converted into compound II. The compound II is methyl 3,4-dimethoxybenzoate 8.

(2) Nitrating Step:

Methyl 3,4-dimethoxybenzoate 8 can be dissolved in acetic acid, then 70% H₂SO₄ and 45% HNO₃ can be added, and then compound IV can be obtained at 25˜150° C. by the nitrating step. The compound IV is methyl 2-nitro-4,5-dimethoxybenzoate 9.

(3) Hydrolysis-Demethylation Step/Esterifying Step:

Methyl 2-nitro-4,5-dimethoxybenzoate 9 can be dissolved in alkali aqueous solution, and the demethylating step can be carried out after the hydrolyzing step at 25° C.˜100° C. The alkali aqueous solution includes water and strong base that is selected from the group consisting of KOH, NaOH and any suitable combination thereof. Reaction formula of the above process can be as follows,

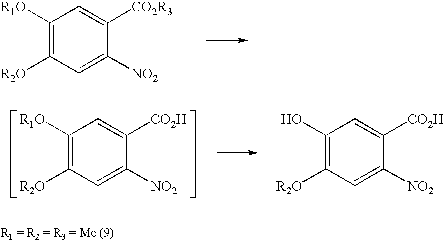

And then the esterifying step can be carried out to obtain compound V. The compound V is methyl 2-nitro-5-hydroxy-4-methoxybenzoate 10.

(4) O-alkylation Step:

Methyl 2-nitro-5-hydroxy-4-methoxybenzoate 10 with 3-morpholinopropoxy chloride can be dissolved in organic solvent or other solvent, weak base is added, and reaction can be carried out at 25˜150° C. to yield compound VI. The compound VI is methyl 2-nitro-4-methoxy-5-(3-morpholinopropoxy)benzoate 4. In addition, the organic solvent can be selected from the group consisting of dimethylformamide, CH₃CN, acetone and any suitable combination thereof, the other solvent can be one of CH₃CN—H₂O and dimethylformamide-H₂O, and the weak base can be selected from the group consisting of K₂CO₃, KHCO₃, NaHCO₃, Na₂CO₃ and any suitable combination thereof.

(5) Reducing Step:

Methyl 2-nitro-4-methoxy-5-(3-morpholinopropoxy)benzoate 4 can be dissolved in alkali solution with Na₂S₂O₄ to be reduced to yield compound VI; or the reducing step can be performed to yield the compound VI, by employing 10% Pd—C as a catalytic agent, using hydrogen gas under a pressure of 30˜60 psi and organic solvent selected from one of ethyl acetate and alcohol at 25˜100° C. The compound VI is methyl 2-amino-5-methoxy-4-(3-morpholinopropoxy)benzoate 5.

(6) Cyclizing Step:

methyl 2-amino-5-methoxy-4-(3-morpholinopropoxy)benzoate 5 can be cyclized to yield compound VII by adding HCO₂NH₄ and HCONH₂ therein at 100˜200° C. The compound VII is 7-methoxy-6-(3-morpholinopropoxy)-3,4-dihydroquinazolin-4-one 6.

(7) One-Pot Reaction (a Chlorinating Step and an Aniline Derivative Substituting Step):

7-methoxy-6-(3-morpholinopropoxy)-3,4-dihydroquinazolin-4-one 6 can be added in organic solvent, then the chlorinating step can be carried out by using 2˜20 equivalents of PCl₅, POCl₃, and then chlorinate that was obtained directly and aniline derivative can be added in alcohol to carry out the aniline derivative substituting step. As such, the product of Gefitinib whose purity is greater than 99.7% can be obtained.

In the method of synthesizing the 6,7-substituted 4-anilino quinazoline of the exemplary embodiment, 3,4-dimethoxybenzoic acid 7 is employed as initial reactant, and the process that the 6,7-substituted 4-anilino quinazoline is synthesized includes the hydrolysis-demethylation step.

In alternative embodiment, in the period of the hydrolysis-demethylation-esterifying step, the reaction temperature can be 25° C.˜150° C., the demethylating step can be selectively carried out on methoxy group corresponding to nitryl of the compound IV with the help of 1˜10 normal AlCl₃ in solvent including toluene, nitrobenzene and CH₂Cl₂. As such, the compound V is obtained, and in other words, methyl 2-nitro-4,5-dimethoxybenzoate 9 is converted into methyl 2-nitro-5-hydroxy-4-methoxybenzoate 10.

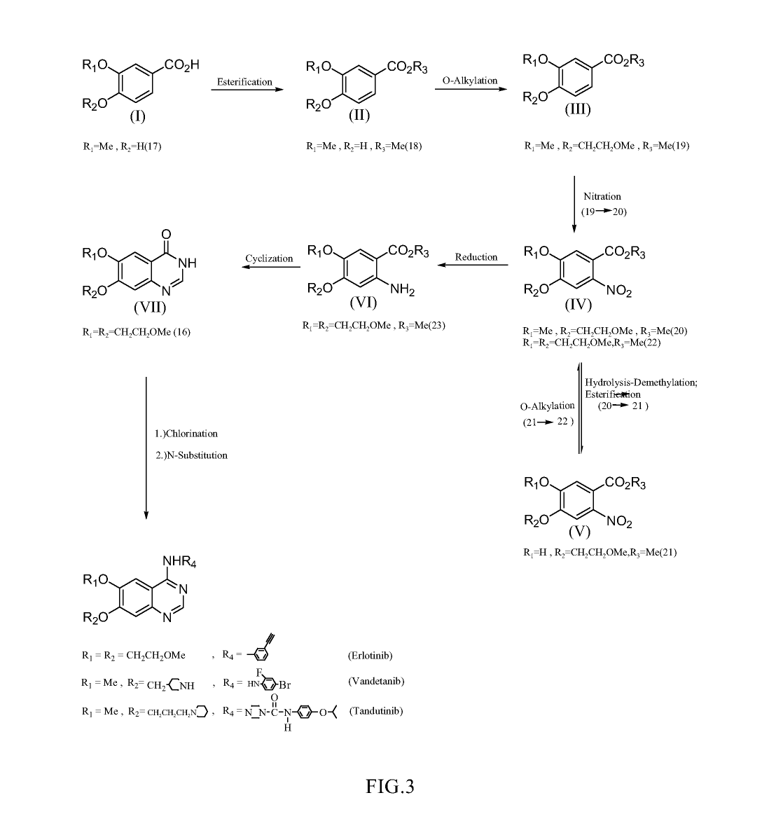

FIG. 3 is a flow chart of a method of synthesizing 6,7-substituted 4-anilino quinazoline according to further another exemplary embodiment of the present invention, showing formulas in different steps. Referring to FIG. 3, in this embodiment, 3-methoxy-4-hydroxybenzoic acid 17 is employed as initial reactant, and the 6,7-substituted 4-anilino quinazoline can be obtained by an esterifying step, an O-alkylation step, a nitrating step, a hydrolysis-demethylation step, an esterifying step, an O-alkylation step, a reducing step, a cyclizing step and an one-pot reaction.

(1) Esterifying Step:

By the Fischer esterifying step or with SOCl₂ in alcohol solvent, 3-methoxy-4-hydroxybenzoic acid 17 can be converted into compound II. The compound II is methyl 3-methoxy-4-hydroxybenzoate 18.

(2) O-alkylation Step:

Methyl 3-methoxy-4-hydroxybenzoate 18 with 2-bromoethyl methyl ether can be dissolved in organic solvent including dimethylformamide, CH₃CN and acetone or in organic solvent and water, and converted into compound III under the condition of weak base or KI, at 50˜150° C. The compound III is methyl 3-methoxy-4-methoxyethoxybenzoate 19.

(3) Nitrating Step:

Methyl 3-methoxy-4-methoxyethoxybenzoate 19 can be dissolved in acetic acid, then 70% H₂SO₄ and 45% HNO₃ can be added, and then compound IV can be obtained at 25˜150° C. by the nitrating step. The compound IV is methyl 2-nitro-5-methoxy-4-methoxyethoxybenzoate 20.

(4) Hydrolysis-Demethylation Step/Esterifying Step:

Methyl 2-nitro-5-methoxy-4-methoxyethoxybenzoate 20 can be dissolved in alkali aqueous solution, and the demethylating step can be carried out after the hydrolyzing step at 25° C.˜100° C. The alkali aqueous solution includes water and strong base that is selected from the group consisting of KOH, NaOH and any suitable combination thereof. Reaction formula of the above process can be as follows,

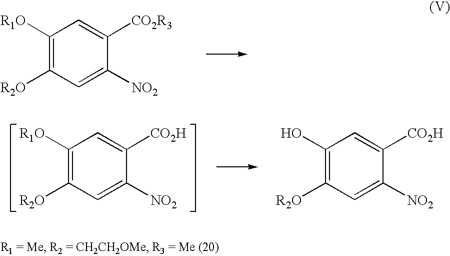

And then the esterifying step can be carried out to obtain compound V. The compound V is methyl 2-nitro-5-hydroxy-4-methoxyethoxybenzoate 21.

(5) O-alkylation Step:

Methyl 2-nitro-5-hydroxy-4-methoxyethoxybenzoate 21 with 2-bromoethyl methyl ether can be dissolved in organic solvent including dimethylformamide, CH₃CN and acetone or in organic solvent and water, and then converted into compound VI, under the condition of weak base or KI, at 50˜150° C. The compound VI is methyl 2-nitro-4,5-dimethoxyethoxybenzoate 22.

(6) Reducing Step:
Methyl 2-nitro-4,5-dimethoxyethoxybenzoate 22 can be dissolved in alkali solution with Na₂S₂O₄ to be reduced to yield compound VI; or the reducing step can be performed to yield the compound VI, by employing 10% Pd—C as a catalytic agent, using hydrogen gas under a pressure of 30˜60 psi and organic solvent selected from one of ethyl acetate and alcohol at 25˜100° C. The compound VI is methyl 2-amino-4,5-dimethoxyethoxybenzoate 23.

(7) Cyclizing Step:

methyl 2-amino-4,5-dimethoxyethoxybenzoate 23 can be cyclized to yield compound VII by adding HCO₂NH₄ and HCONH₂ therein at 100˜200° C. The compound VII is 6,7-bis(2-methoxyethoxy)quinazolin-4-one 16.

(8) One-Pot Reaction (a Chlorinating Step and an Aniline Derivative Substituting Step):

6,7-bis(2-methoxyethoxy)quinazolin-4-one 16 can be added in organic solvent, then be chlorinated by using 2˜20 equivalents of PCl₅, POCl₃ and SOCl₂, and then chlorinate that was obtained directly and aniline derivative can be added in alcohol to carry out the aniline derivative substituting step. As such, the product of Erlotinib whose purity is greater than 99.7% can be obtained.

In the method of synthesizing the 6,7-substituted 4-anilino quinazoline of the exemplary embodiment, 3-methoxy-4-hydroxybenzoic acid 17 is employed as initial reactant, and the process of that the 6,7-substituted 4-anilino quinazoline, such as Erlotinib, is synthesized, including the hydrolysis-demethylation step and the esterifying step. In addition, if the method does not include the hydrolysis-demethylation step and the esterifying step, the synthesized 6,7-substituted 4-anilino quinazoline can be Vandetanib and Tandutinib.

In alternative embodiment, in the period of the hydrolysis-demethylation step and the esterifying step, the reaction temperature can be 25° C.˜150° C., the demethylating step can be selectively carried out on methoxy group corresponding to nitro group of the compound IV with about 1˜10 equivalents of AlCl₃ in solvent including toluene, nitrobenzene and CH₂Cl₂. As such, the compound V is obtained, and in other words methyl 2-nitro-5-methoxy-4-methoxyethoxybenzoate 20 is converted into methyl 2-nitro-5-hydroxy-4-methoxyethoxybenzoate 21.

In summary, the methods of synthesizing the 6,7-substituted 4-anilino quinazoline of the present invention have relatively high yield. The overall yield can be 30-42%. Furthermore, the above methods have some advantages, such as easy to recycle and purify, and the purity of the final product synthesized by the above methods is not less than 99.7%. In addition, the one-pot reaction is used as the final step of the method for preparing the product and the initial reactant has low cost. Therefore, the method of the present invention has a commercial advantage.

The above description is given by way of example, and not limitation. Given the above disclosure, one skilled in the art could devise variations that are within the scope and spirit of the invention disclosed herein, including configurations ways of the recessed portions and materials and/or designs of the attaching structures. Further, the various features of the embodiments disclosed herein can be used alone, or in varying combinations with each other and are not intended to be limited to the specific combination described herein. Thus, the scope of the claims is not to be limited by the illustrated embodiments.

The condition and intermediate of each of the steps will be described detailedly as follows.

(I) O-alkylation Step

### (1) Preparation of Methyl 4-methoxy-3-(3-morpholinopropoxy)benzoate 3

Methyl 3-hydroxy-4-methoxybenzoate 2 (25.5 g, 0.1401 mol) is dissolved in CH₃CN (255 ml), and 3-morpholinopropoxy chloride (27.54 g, 0.1684 mol) is added. K₂CO₃ (32.78 g, 0.2375 mol) is dissolved in water (76.5 ml), and mixed with the above solution to react at 80° C. for 3 hours. After water (255 ml) is added to the reaction mixture, the reaction mixture is extracted by ethyl acetate (255 ml) for twice, and extracts of ethyl acetate layer are combined, to be dried by MgSO₄, filtered and concentrated to obtain yellowish solid compound 3 (43.5 g, 100%). ¹H-NMR (CDCl₃) spectrum: 1.95 (m, 2H), 2.39 (brs, 4H), 2.46 (m, 2H), 3.62 (m, 4H), 3.78 (s, 3H), 3.82 (s, 3H), 4.12 (m, 2H), 6.78 (dd, 1H, J=8.8 Hz, J=2.0 Hz), 7.48 (s, 1H), 7.58 (dd, 1H, j=8.8 Hz, J=2.0 Hz).

### (2) Preparation of Methyl 2-nitro-4-methoxy-5-(3-morpholinopropoxy)benzoate 4

Methyl 2-nitro-5-hydroxy-4-methoxyethoxybenzoate 21 (1 g, 0.0044 mol) is dissolved in dimethylformamide (10 ml), and 3-morpholinopropoxy chloride (0.83 g, 5.269 mmol) is added. K₂CO₃ (1.21 g, 8.768 mmol) is added to react at 50-100° C. for 1 hour. And then, after water (20 ml) is added, the reaction mixture is extracted by ethyl acetate (20 ml) for three times, and extracts of ethyl acetate layer are combined, to be dried by MgSO₄, filtered and concentrated to obtain yellowish solid compound 4 (1.5 g, 96.18%). ¹H-NMR (CDCl₃) spectrum: 2.02 (t, 2H, J=6.8 Hz), 2.42 (brs, 4H), 2.51 (m, 2H), 3.68 (m, 4H), 3.87 (s, 3H), 3.92 (s, 3H), 4.16 (t, 2H, J=6.8 Hz), 7.07 (s, 1H), 7.42 (s, 1H).

### (3) Preparation of Ethyl 3,4-dimethoxyethoxybenzoate 13

Ethyl 3,4-dihydroxy benzoate 12 (5 g, 27.45 mmol) is placed in a two-neck bottle of 250 ml, N₂ is added at room temperature, and acetone (100 ml), potassium carbonate (9.48 g, 68.63 mmol), potassium iodide (0.5 g) and 2-Bromoethyl methyl ether (7.84 ml, 82.35 mmol) are added. Then heat refluxing is carried out at 60° C. for 19 hours. After the reaction is completed, the resultant of reaction is cooled at 5° C. and stirred for 30 minutes, then filtered and concentrated to dry. The solid is dried by oil-less pump for 22 hours to obtain khaki compound 13 (8.19 g, 100%). ¹H-NMR (CDCl₃) spectrum: 1.32 (t, 3H, J=7 Hz), 3.41 (s, 6H), 3.76 (m, 4H), 4.15 (m, 4H), 4.29 (q, 2H, J=7 Hz), 6.85 (d, 1H, J=8.4 Hz), 7.53 (dd, 1H, J=8.4 Hz, J=2.3 Hz), 7.53 (m, 1H), 7.63 (dd, 1H, J=8.4 Hz, J=2.3 Hz).

### (4) Preparation of Methyl 3-methoxy-4-methoxyethoxybenzoate 19

Similar to the above process, methyl 3-methoxy-4-hydroxybenzoate 18 is employed as initial reactant to prepare compound 19 in 97.8% yield as butter color solid. ¹H-NMR (CDCl₃) spectrum: 3.41 (s, 3H), 3.77 (m, 2H), 3.85 (s, 3H), 3.87 (s, 3H), 4.19 (m, 2H), 6.87 (d, 1H, J=8.48 Hz), 7.51 (s, 1H), 7.61 (dd, 1H, J=8.48 Hz, J=2.0 Hz)

### (5) Preparation of Methyl 2-nitro-4,5-dimethoxyethoxybenzoate 22

Similar to the above process, methyl 2-nitro-5-hydroxy-4-methoxyethoxybenzoate 21 is employed as initial reactant to prepare compound 22 in 95.2% yield as butter color solid. ¹H-NMR (CDCl₃) spectrum: 3.34 (s, 6H), 3.76 (m, 4H), 3.86 (s, 3H), 4.21 (m, 4H), 7.08 (s, 1H), 7.48 (s, 1H).

(II) Nitrating Step

### (1) Preparation of methyl 2-nitro-4-methoxy-5-(3-morpholinopropoxy)benzoate 4

Methyl 4-methoxy-3-(3-morpholinopropoxy)benzoate 3 (43.5 g, 0.1408 mol) is dissolved in acetic acid (117 ml) at normal temperature, after moving to an environment with 5° C. for ten minutes, HNO₃ (21.75 ml, 45.5%) is added to react for 30 minutes, H₂SO₄ (44 ml, 70%) is added, and after cooled to room temperature, reaction is carried out for 2 hours. After reaction is completed, ice water (300 ml) is added at 5° C., water (170 ml) is used to wash a bottle with a rounded bottom, alkalinity is adjusted by adding NaOH (280 ml, 50%), stirring is carried out for 1 hours at a temperature less than 5° C., and extraction is performed by using ethyl acetate (770 ml) for twice. Extracts of ethyl acetate layer are combined, to be dried by MgSO₄, filtered, concentrated and dried for 16 hours to obtain daffadilly solid compound 4 (46 g, 92.3%).

### (2) Preparation of Methyl 2-nitro-4,5-dimethoxybenzoate 9

Similar to the above process, methyl 3,4-dimethoxybenzoate 8 is employed as initial reactant to prepare compound 9 in 95% yield as yellow solid. ¹H-NMR (CDCl₃) spectrum: 3.84 (s, 3H), 3.99 (s, 6H), 7.27 (s, 1H), 7.57 (s, 1H).

### (3) Preparation of Ethyl 2-nitro-4,5-dimethoxyethoxybenzoate 14

Similar to the above process, ethyl 3,4-dimethoxyethoxybenzoate 13 is employed as initial reactant to prepare compound 14 in 81% yield as brown liquid. ¹H-NMR (CDCl₃) spectrum: 1.31 (t, 2H, J=7 Hz), 3.41 (s, 6H), 3.77 (m, 4H), 4.20 (m, 4H), 4.33 (q, 2H, J=7 Hz), 7.08 (s, 1H), 7.47 (s, 1H)

### (4) Preparation of Methyl 2-nitro-5-methoxy-4-methoxyethoxybenzoate 20

Similar to the above process, methyl 3-methoxy-4-methoxyethoxybenzoate 19 is employed as initial reactant to prepare compound 20 in 93% yield as yellow solid. ¹H-NMR (CDCl₃) spectrum: 3.42 (s, 3H), 3.78 (m, 2H), 3.87 (s, 3H), 3.93 (s, 3H), 4.22 (m, 2H), 7.04 (s, 1H), 7.48 (s, 1H)

(III) Hydrolysis-Demethylation Step/Esterifying Step

### (1) Preparation of Methyl 2-nitro-5-hydroxy-4-methoxybenzoate 10

Methyl 2-nitro-4,5-dimethoxybenzoate 9 (3 g, 0.0124 mol) is added to KOH solution (20 ml, 20%), reaction is carried out at 100° C. for 5 hours. After cooled to room temperature, 1N HCl (50 ml) is added while stirred, extraction is performed by using ethyl acetate (30 ml) for three times. Extracts of ethyl acetate layer are combined, to be dried by MgSO₄, filtered and concentrated to obtain yellowish solid compound (2.8 g). Methanol (28 ml) is added, concentrated sulphuric acid (1.0 ml) is added at room temperature, nitrogen gas is added and heat refluxing is carried out for 6 hours. After cooled to room temperature, water (28 ml) is added to terminate the reaction, alkalinity is adjusted by adding NaHCO_{3(Sat)} (145 ml), and then extraction is performed by using ethyl acetate (28 ml) for three times. Extracts of ethyl acetate layer are combined, to be dried by MgSO₄, filtered and concentrated to obtain solid compound 10 with butter color (2.64 g, 93.5%). ¹H-NMR (CDCl₃) spectrum: 3.96 (s, 3H), 7.06 (s, 1H), 7.55 (s, 1H).

### (2) Preparation of Methyl 2-nitro-5-hydroxy-4-methoxyethoxybenzoate 21

Similar to the above process, methyl 2-nitro-5-methoxy-4-methoxyethoxybenzoate 20 is employed as initial reactant to prepare compound 21 in 94% yield as yellow solid. ¹H-NMR (CDCl₃) spectrum: 3.44 (s, 3H), 3.75 (m, 2H), 3.87 (s, 3H), 4.22 (m, 2H), 7.08 (s, 1H), 7.58 (s, 1H).

(IV) Demethylating Step

### Preparation of Methyl 2-nitro-5-hydroxy-4-methoxyethoxybenzoate 21

Aluminum chloride (11.68 g, 87.65 mmol) is placed in a single neck bottle of 500 ml, N₂ is added at room temperature, CH₂Cl₂ (50 ml) is added, methyl 2-nitro-5-methoxy-4-methoxyethoxybenzoate 20 (5 g, 17.53 mmol) are respectively added slowly, and after moved to an environment with 40° C., heat refluxing is carried out for 1 hour. After cooled to 5° C., ethyl acetate (50 ml) is added, water (50 ml) is added to terminate the reaction, CH₂Cl₂ is removed by concentration, extraction is performed by using ethyl acetate (50 ml×2), organic layers are combined to be washed by water (100 ml), and the organic layers is dried by adding anhydrous MgSO₄, filtered and concentrated to obtain yellow solid compound 21 (4.78 g, 100%).

(V) Reducing Step

### (1) Preparation of Methyl 2-amino-5-methoxy-4-(3-morpholinopropoxy)benzoate 5

Methyl 2-nitro-4-methoxy-5-(3-morpholinopropoxy)benzoate 4 (46 g, 0.1299 mol) is dissolved entirely in ethyl acetate (430 ml) at 50° C., and then cooled to normal temperature. Pd—C (4.6 g, 10%) is placed in a beaker, and ethyl acetate (30 ml) is added slowly. The mixture of Pd—C and ethyl acetate is added to the above solution, and hydrogen gas under a pressure of 50 psi is added to react for 3 hours. After the reaction is completed, ethyl acetate (230 ml) is used to wash for once. After filtered and concentrated, wheat solid compound 5 (41.6 g, 98.8%) is obtained. ¹H-NMR (CDCl₃) spectrum: 1.93 (m, 2H), 2.44 (m, 4H), 2.51 (m, 2H), 3.69 (m, 4H), 3.80 (s, 6H), 3.96 (m, 2H), 5.55 (brs, 1H), 6.09 (s, 1H), 7.30 (s, 1H).

### (2) Preparation of Ethyl 2-amino-4,5-dimethoxyethoxybenzoate 15

Ethyl 2-nitro-4,5-dimethoxyethoxybenzoate 14 (7 g, 20.40 mmol) is placed in a two-neck bottle of 500 ml, THF (30 ml), water (140 ml) and NH₄OH (4 ml, 28˜30%) are added, then sodium hydrosulfite (3.88 g, 28.04 mmol) is added, and heat refluxing is carried out for 2 hours. Conc.HCl (4 ml) is added to continue heat refluxing for 2 hours. After reaction is completed and cooled to 5° C., NaOH (18 ml, 20%) is added to adjust pH>9. Extraction is performed by using ethyl acetate (200 ml×2), organic layers are combined to be washed by water (200 ml), and the organic layers is dried by adding anhydrous MgSO₄, filtered and concentrated to obtain brown liquid compound 15 (5.34 g, 85.1%). ¹H-NMR (CDCl₃) spectrum: 1.33 (t, 2H, J=7.1 Hz), 3.41 (s, 6H), 3.68˜3.71 (m, 4H), 4.04˜4.25 (m, 4H), 4.26 (q, 2H, J=7.1 Hz), 6.14 (s, 1H), 7.42 (s, 1H).

### (3) Preparation of Methyl 2-amino-4,5-dimethoxyethoxybenzoate 23

Similar to the above process, methyl 2-nitro-4,5-dimethoxyethoxybenzoate 22 is employed as initial reactant to prepare. compound 23 in 93% yield as yellow solid. ¹H-NMR (CDCl₃) spectrum: 3.41 (s, 6H), 0.68˜3.74 (m, 4H), 3.82 (s, 3H), 4.03˜4.10 (m, 4H), 5.55 (brs, 1H), 6.12 (s, 1H), 7.38 (s, 1H).

(XI) Cyclizing Step

### (1) Preparation of 7-methoxy-6-(3-morpholinopropoxy)-3,4-dihydroquinazolin-4-one

Methyl 2-amino-5-methoxy-4-(3-morpholinopropoxy)benzoate 5 (41.6 g, 0.1284 mol) is placed in a bottle with a rounded bottom, HCONH₂ (108 ml) and HCO₂NH₄ (9.8 g, 0.0762 mmol) are added to react for 3 hours at 170° C. After cooled to normal temperature, filter and dried, ice water (83.2 ml) is used to wash to obtain yellowish-white solid compound 6 (14.8 g, 71.1%). ¹H-NMR (DMSO) spectrum: 2.07 (m, 2H), 2.58 (brs, 4H), 2.61 (t, 2H, J=6.8 Hz), 3.71 (m, 4H), 3.98 (s, 3H), 4.18 (t, 2H, J=6.8 Hz), 7.15 (s, 1H), 7.59 (s, 1H), 8.00 (s, 1H).

### (2) Preparation of 6,7-bis(2-methoxyethoxy)quinazolin-4-one 16

Similar to the above process, ethyl 2-amino-4,5-dimethoxyethoxybenzoate 15 or methyl 2-amino-4,5-dimethoxyethoxybenzoate 23 is employed as initial reactant to prepare. compound 16 in 77% yield as off-white solid. ¹H-NMR (CDCl₃) spectrum: 3.71 (s, 6H), 3.82 (brs, 4H), 3.90 (brs, 4H), 6.26 (s, 1H), 6.57 (s, 1H), 7.58 (s, 1H)

(XII) One-Pot Reaction

(1) Preparation of Gefitinib

7-methoxy-6-(3-morpholinopropoxy)-3,4-dihydroquinazolin-4-one (29.12 g, 0.0913 mol) is dissolved in toluene, Et₃N (19 ml, 0.1366 mol) is added at 5° C., and after POCl₃ (17.8 ml, 0.1824 mol) is added, reaction is carried out for 3 hours at 70° C. 3-chloro-4-fluoroaniline (15.9 g, 0.1093 mol) mixed into the isopropyl alcohol (10 ml) is added to the above reaction solution, and then stirring is carried out for 1 hour at 70° C. Wheat solid compound is obtained by filter, water (380 ml) is added to dissolve the solid compound entirely, NaOH (30 ml, 20%) is added, and after stirred for 1 hour, filter is carried out. After dissolved solid and filtered, Gefitinib (25.65 g, 62.86%) that is white solid compound is obtained, whose purity determined by HPLC is greater than 99.9%. ¹H-NMR (DMSO) spectrum: 2.21 (brs, 2H), 2.84 (brs, 4H), 2.92 (brs, 2H), 3.80 (brs, 4H), 3.99 (s, 3H), 4.28 (brs, 2H), 7.15 (s, 1H), 7.24 (t, 1H, J=8.9 Hz), 7.71 (m, 2H) 8.00 (m, 1H), 8.44 (s, 1H)

(2) Preparation of Erlotinib

6,7-bis(2-methoxyethoxy)quinazolin-4-one 16 (0.53 g, 1.80 mmol) is placed in a two-neck bottle of 50 ml, N₂ is added at room temperature, toluene (5.3 ml) and triethylamine(0.39 ml=0.28 g, 2.77 mmol), phosphorus oxychloride(0.57 ml=0.94 g, 6.12 mmol) is added and stirred for 10 minutes, and at 65° C. N₂ is added and reaction is carried out for 3 hours. 3-aminophenylacetylen (0.22 g, 1.84 mmol) dissolved in isopropanol (1 ml) is added to the above reaction solution to continue reaction for 2.5 hours. After the reaction is completed, Erlotinib (0.5 g, 64%) that is white solid compound is obtained by cooling, filter and solid drying (in oven at 65° C.), whose purity determined by HPLC is greater than 99.7%. ¹H-NMR (DMSO) spectrum: 3.35 (s, 6H), 3.77 (brs, 4H), 4.28 (s, 1H), 4.32 (brs, 2H), 4.38 (brs, 2H), 7.41 (m, 2H), 7.49 (m, 1H), 7.78 (m, 1H), 7.87 (s, 1H), 8.44 (s, 1H), 8.85 (s, 1H), 11.56 (s, 1H).

What is claimed is:

1. A method of synthesizing 6,7-substituted 4-anilino quinazoline, the 6,7-substituted 4-anilino quinazoline represented by formula as follows, 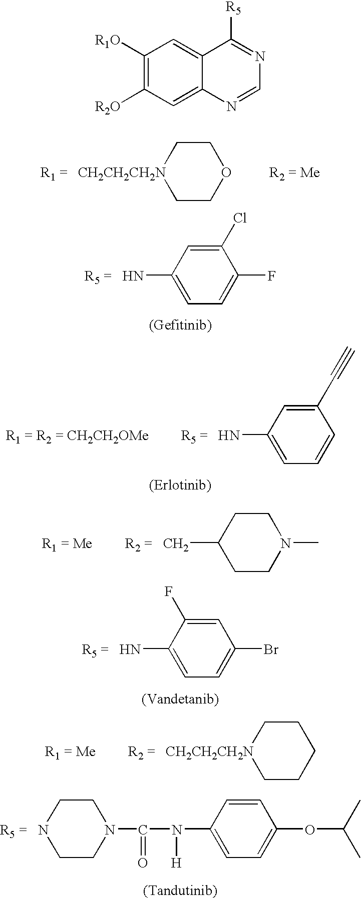
the method comprising:
(a) employing 3,4-substituted benzoic acid as an initial reactant, and a first esterifying step performed on 3,4-substituted benzoic acid to yield alkyl 3,4-substituted benzoate, wherein each substituted group of the 3,4-substituted benzoic acid or the alkyl 3,4-substituted benzoate is one of an alkyl group and a hydroxyl group, and oxygen in the substituted groups is defined as a first oxygen and a second oxygen correspondingly;
(b) nitrating the alkyl 3,4-substituted benzoate to yield alkyl 2-nitro-4,5-substituted benzoate;
(c) reducing the alkyl 2-nitro-4,5-substituted benzoate to yield alkyl 2-amino-4,5-substituted benzoate;
(d) cyclizing the alkyl 2-amino-4,5-substituted benzoate to yield 6,7-substituted quinazolin-4-one; and
(e) performing a one-pot reaction on the 6,7-substituted quinazolin-4-one to yield 6,7-substituted 4-anilino quinazoline, wherein the one-pot reaction comprises a chlorinating step and an aniline derivative substituting step.

2. The method of synthesizing 6,7-substituted 4-anilino quinazoline according to claim 1, further comprises a first O-alkylation step on the first oxygen before the nitrating step, wherein the first O-alkylation step is performed by using water, weak base substance, organic solvent and a small quantity of catalytic agent at 50˜150° C., the weak base substance is selected from the group consisting of K₂CO₃, KHCO₃, NaHCO₃, Na₂CO₃ and any combination thereof, and the organic solvent is selected from the group consisting of CH₃CN, dimethylformamide, acetone and any combination thereof.

3. The method of synthesizing 6,7-substituted 4-anilino quinazoline according to claim 1, further comprises a hydrolysis-demethylation step, a second esterifying step and a second O-alkylation step on the second oxygen before the nitrating step; wherein the hydrolysis-demethylation step is performed in aqueous solution with strong base substance or aqueous solution having organic solvent with strong base substance at 25° C.˜100° C., and the strong base is selected from the group consisting of KOH, NaOH and any combination thereof,
the second esterifying step is performed by using inorganic acid with C1˜C6 alcohol solvent or SOCl₂ with C1˜C6 alcohol solvent at 25˜100° C.; and
the second O-alkylation reaction on the second oxygen is performed under the condition of introducing weak base substance, organic solvent, water and a small quantity of catalytic agent at 50˜150° C., the weak base substance is selected from the group consisting of K₂CO₃, KHCO₃, NaHCO₃, Na₂CO₃ and any combination thereof, and the organic solvent is selected from the group consisting of CH₃CN, dimethylformamide, acetone and any combination thereof.

4. The method of synthesizing 6,7-substituted 4-anilino quinazoline according to claim 1, further comprises a demethylating step and a second esterifying step before the nitrating step,
wherein the demethylating step is performed by using organic solvent and 1˜10 normal AlCl₃ at 25˜150° C. and the organic solvent is selected from the group consisting of toluene, nitrobenzene, CH₂Cl₂ and any combination thereof; and
wherein the second esterifying step is performed by using inorganic acid with C1˜C6 alcohol solvent or SOCl₂ with C1˜C6 alcohol solvent at 25˜100° C.

5. The method of synthesizing 6,7-substituted 4-anilino quinazoline according to claim 1, wherein the first esterifying step is performed under inorganic acid with C1˜C6 alcohol solvent or SOCl₂ with C1˜C6 alcohol solvent at 25˜100° C.

6. The method of synthesizing 6,7-substituted 4-anilino quinazoline according to claim 1, wherein the nitrating step is performed by using acetic acid, sulphuric acid and nitric acid.

7. The method of synthesizing 6,7-substituted 4-anilino quinazoline according to claim 1, wherein the reducing step is performed in alkali solution with Na₂S₂O₄ or the reducing step is performed by using organic solvent, 10% Pd—C as a catalytic agent and hydrogen gas under a pressure of 30˜60 psi at 25˜100° C., and the organic solvent is ethyl acetate or C1˜C6 alcohol.

8. The method of synthesizing 6,7-substituted 4-anilino quinazoline according to claim 1, wherein the cyclizing step is performed in HCO₂NH₄ and HCONH₂ at 80˜200° C.

9. The method of synthesizing 6,7-substituted 4-anilino quinazoline according to claim 1, wherein the chlorizing step is performed in organic solvent with PCl₅, POCl₃ and SOClₐₜ 25˜160° C., and the organic solvent is selected from the group consisting of benzene, toluene, dimethylfomamide and any combination thereof.

10. The method of synthesizing 6,7-substituted 4-anilino quinazoline according to claim 1, wherein the aniline derivative substituting step is performed in C1˜C4 alcohol with aniline derivative compound.
